(19) United States
(12) Patent Application Publication
(10) Pub. No.: US 2016/0174883 A1
(43) Pub. Date: Jun. 23, 2016

(54) METHOD FOR THE DIAGNOSIS AND MONITORING OF CHEMOTHERAPY-INDUCED PERIPHERAL NEUROPATHY

(71) Applicant: IMPETO MEDICAL, Paris (FR)

(72) Inventors: Jean-Henri Calvet, Paris (FR); Philippe Brunswick, Paris (FR)

(73) Assignee: IMPETO MEDICAL, Paris (FR)

(21) Appl. No.: 14/575,778

(22) Filed: Dec. 18, 2014

(51) Int. Cl. A61B 5/145; A61K 31/555; A61K 31/337; A61B 5/1477; A61B 5/053

(52) U.S. Cl. CPC A61B 5/14517; A61B 5/1477; A61B 5/0537; A61K 31/337; A61K 31/555; A61B 2562/0209

(57) ABSTRACT

The present disclosure relates to a method of diagnosing a CIPN in a subject, the method including assessing the sudomotor function of the subject. Also disclosed are methods of monitoring progression of the disease, as well as methods of treating the condition.

### BACKGROUND

Modern cancer therapy prolongs patients life but commonly increases incidence of treatment-related complications. One of such adverse effect is a neurotoxicity, which usually translates as peripheral neuropathies: chemotherapy-induced peripheral neuropathy (CIPN) is a progressive, enduring, and often irreversible condition featuring pain, numbness, tingling and sensitivity to cold in the hands and feet (sometimes progressing to the arms and legs), and esophagus, that afflicts between 30 and 40 percent of patients undergoing chemotherapy (Park et al., CA Cancer J Clin, 63: 419-437, 2013). These effects may lead to disability and worsening of life quality in the absence of tumor progression and it represents a serious dose-limiting side effect. In addition, the development of the neurotoxic syndrome can interfere with optimal drug dosing, delay sequencing of therapy, or necessitate the discontinuation of treatment.

CIPN may result from the use of numerous chemotherapeutic agents. Chemotherapy combinations with higher incidences include notably those that involve platinum drugs, vinca alkaloids, bortezomib, and/or taxanes. However, no neuroprotective treatment exists. In addition, there are no established agents recommended for the prevention of CIPN in patients with cancer undergoing treatment with neurotoxic agents (Hershman et al., J Clin Oncol, 32(18): 1941-1967, 2014).

Early detection of CIPN is thus crucial for designing or adapting an appropriate treatment. However, the diagnosis of CIPN may present a diagnostic dilemma due to the large number of potential toxic etiologies and conditions, which may mimic some of the clinical features. Diagnosis of CIPN currently relies on the Total Neuropathy Score (TNSc) (Park et al., 2013). Unfortunately, the TNSc is time-consuming to perform and is rarely performed by the physician who prescribes chemotherapy. In addition, since it is based on the assessment of several clinical parameters by the neurologist, it is tainted by an element of subjectivity which may affect the results, in particular at the onset of the condition. Finally, it cannot be used in asymptomatic patients. There is thus still a need for a reliable method for detecting small-fiber neuropathies in patients receiving chemotherapies.

### SUMMARY

In a first aspect, the present invention relates to a method of diagnosing a CIPN in a subject, said method comprising the step of assessing the sudomotor function of said subject. In a preferred embodiment, a new technology measuring the capacity of the sweat glands to release chloride ions in response to an electrochemical stimulus is used to evaluate the likelihood of CIPN in said subject. The invention provides a non-invasive measurement that is quick and easy to implement on the subject.

The method according to the invention is carried out with a system comprising an anode and a cathode, intended to be placed on different regions of the subject body, and an adjustable DC source, which is controlled in order to feed the anode with a DC current. In a further preferred embodiment, the method comprises the steps consisting of:
applying DC voltage pulses of varying voltage values to the anode for given durations allowing the stabilization of electrochemical phenomena in the body in the vicinity of the electrodes,
collecting data representative of the current between the anode and the cathode, and of the potentials of the anode and the cathode, for the different DC voltages,
from said data, computing data representative of the electrochemical skin conductance of the subject.

The electrochemical skin conductance value at a given voltage applied on the anode may be determined as the ratio between the current through the anode and the cathode and the voltage difference between the anode and the cathode. The computed data relative to the electrochemical skin conductance values of the patient may include the difference and/or the ratio between two electrochemical skin conductance values of the patient for two different voltage values applied to the anode.

In additional embodiments, the present invention includes a system for diagnosing a patient, with a view to detecting CIPN, comprising:
an anode and a cathode, intended to be placed on different regions of the patient body,
an adjustable DC source, which is controlled in order to feed the anode with pulses of a DC current of varying voltage values, for given durations allowing the stabilization of electrochemical phenomena in the body in the vicinity of the electrodes,
a measuring circuit, designed to collect data representative of the current between the anode and the cathode, and of the potentials of the anode and the cathode, for the different DC voltages,
   wherein the system further comprise a computing circuit, designed to compute data representative of the electrochemical skin conductance of the patient and to reconcile said data with reference data obtained in the same conditions on patients identified as suffering or not from CIPN.

In another aspect, the invention relates to a method of determining the likelihood for a cancer patient treated by chemotherapy, to develop symptoms of CIPN. In particular, a decreased electrochemical skin conductance indicates that said patient is likely to develop symptoms of CIPN. According to another aspect, the invention provides a method for monitoring the progression of CIPN. In particular, according to a preferred embodiment, the invention provides a method for prognosing CIPN in a cancer patient treated by chemotherapy, wherein the electrochemical skin conductance of the patient is indicative of the outcome of CIPN in said patient.

In yet another aspect, the invention provides a method for adapting a chemotherapy treatment in a cancer patient, wherein said chemotherapy maybe reduced or suppressed if said patient is likely to develop CIPN. Alternatively, said treatment may be continued or increased if the patient is not likely to develop CIPN.

### BRIEF DESCRIPTION OF THE DRAWINGS

FIG. 1 shows a system for diagnosing a patient according to the invention.

FIG. 2 shows the main steps carried out in the diagnosis method provided by the invention.

FIG. 3 shows the distribution of the TNSc score at baseline and maximum value during follow-up (n=71). Median value of TNSc at baseline=2. Median value of maximum TNSc score=6.

FIG. 4 displays a comparison of first and lowest hands ESC values during the follow-up according to the treatment received.

FIG. 5 presents a comparison of first and lowest feet ESC values according to the treatment received.

FIG. 6 shows a comparison of first and lowest hands and feet ESC values among asymptomatic patients (i.e. patients with a null score for the first and second question of the TNSc) (n=54 among 71).

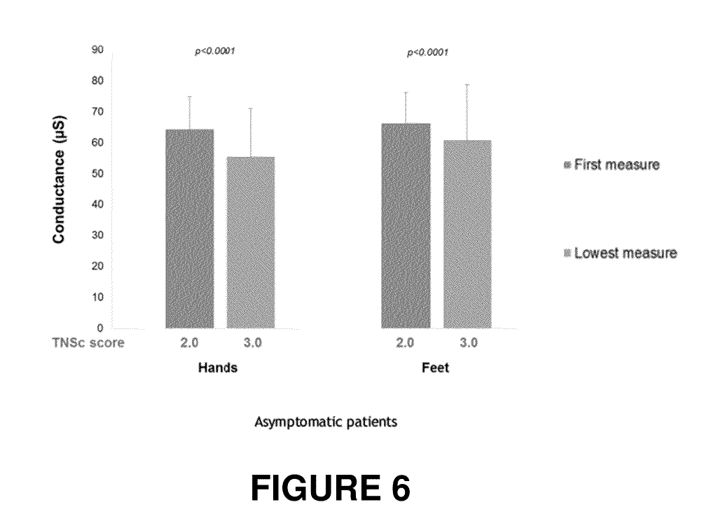

FIG. 7 shows a comparison of hands and feet conductance according to the TNSc score among patients receiving oxaliplatin (n=28). Correlation between feet ESC and TNSc=−0.4 (p<0.0001).

### DETAILED DESCRIPTION

Definitions:

Unless specifically defined, all technical and scientific terms used herein have the same meaning as commonly understood by a skilled artisan in chemistry, biochemistry, cellular biology, molecular biology, and medical sciences.

As used herein “chemotherapeutic agent” or “chemotherapy agent” or “antineoplastic agent” refers to an agent that reduces, prevents, and/or delays the growth of metastases or neoplasms, or kills neoplastic cells directly by necrosis or apoptosis in a pharmaceutically-effective amount, to reduce, prevent, and/or delay the growth of metastases or neoplasms in a subject with neoplastic disease.

“Chemotherapy” refers to treatments using chemotherapeutic agents, chemotherapy agents, or antineoplastic agents.

“Chemotherapy-induced peripheral neuropathy” is a toxic neuropathy that results from the direct injury of the peripheral nervous system by a chemotherapeutic agent(s). CIPN can be acute or chronic. CIPN can be sensory, motor, autonomic, or a mixture of any of the three classes.

The term “decreased”, as used herein, refers to the level of sudomotor function, in particular as assessed by the electrochemical skin conductance, of a subject at least 1-fold (e.g. 1, 2, 3, 4, 5, 10, 20, 30, 40, 50, 60, 70, 80, 90, 100, 1000, 10,000-fold or more) lower than its reference value. “Decreased”, as it refers to the level of the sudomotor function of a subject, notably of electrochemical skin conductance of said subject, signifies also at least 5% lower (e.g. 5%, 6%, 7%, 8%, 9%, 10%, 15%, 20%, 25%, 30%, 35%, 40%, 45%, 50%, 55%, 60%, 65%, 70%, 75%, 80%, 85%, 90%), 95%), 99%), or 100%) than the level in the reference sample or with respect to the reference value for said sudomotor function, and particularly of electrochemical skin conductance.

As used herein, “diagnosis” or “identifying a subject having” refers to a process of determining if an individual is afflicted with a disease or ailment (e.g., CIPN). CIPN is diagnosed for example by assessing the activity of the sweat glands of the patient.

As used herein, “identifying” as it refers to a subject that has a condition refers to the process of assessing a subject and determining that the subject has a condition, for example, is affected by CIPN.

The term “increased”, as used herein, refers to the level of sudomotor function, in particular as assessed by the electrochemical skin conductance, of a subject at least 1-fold (e.g. 1, 2, 3, 4, 5, 10, 20, 30, 40, 50, 60, 70, 80, 90, 100, 1000, 10,000-fold or more) greater than its reference value. “Increased”, as it refers to the level of sudomotor function, notably of electrochemical skin conductance, of a subject, signifies also at least 5% greater (e.g. 5%, 6%, 7%, 8%, 9%, 10%, 15%, 20%, 25%, 30%, 35%, 40%, 45%, 50%, 55%, 60%, 65%, 70%, 75%, 80%, 85%, 90%), 95%), 99%), or 100%) than the level in the reference sample or with respect to the reference value for said sudomotor function, and particularly of electrochemical skin conductance.

As used herein, “monitoring disease progression” refers to a process of determining the severity or stage of a disease in an individual afflicted with the disease or ailment (e.g., CIPN).

As used herein, “neuropathy” refers to any disorder of the central and/or peripheral nervous systems. It can be caused by various causes, such as e.g., traumatic injury, illness or degenerative spine conditions. “Peripheral neuropathy” as used herein refers to disorders of the peripheral nervous system. “Neuropathic pain” is the intractable pain caused by dysfunction in the peripheral or central nervous system.

The term “reference value”, as used herein, refers to the sudomotor function, preferably as assessed by measuring the electrochemical skin conductance, under consideration in a reference population. A “reference population”, as used herein, means a population comprising subjects, preferably two or more subjects, known to be free of CIPN or, alternatively, from the general population. The suitable reference levels of sudomotor activity, notably of electrochemical skin conductance, can be determined by measuring the levels of said sudomotor function, and particularly of electrochemical skin conductance, in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual value such as, for example, a value obtained from the subject being tested, but at an earlier point in time. The reference value can be based on a large number of samples, such as from population of subjects of the chronological age matched group, or based on a pool of samples including or excluding the sample to be tested.

A “subject” which may be subjected to the methodology described herein may be any of mammalian animals including human, dog, cat, cattle, goat, pig, swine, sheep and monkey. A human subject can be known as a patient.

As used herein, “sudomotor function” refers to the stimulation of the sweat glands. Sudomotor function or sweat gland activity is controlled by a division of the sympathetic system, the post-sympathetic cholinergic fibers, which are classified as C-fibers, but also by the microcirculatory vessels (capillaries) which mediate vasodilation (hyperemia), increased skin temperature and sweat output.

As used herein, the terms “treat,” treating,” “treatment,” and the like refer to reducing or ameliorating the symptoms of a disorder (e.g., cancer, CIPN, etc.) and/or symptoms associated therewith. It will be appreciated that, although not precluded, treating a disorder or condition does not require that the disorder, condition or symptoms associated therewith be completely eliminated.

As used herein “treating” a disease in a subject or “treating” a subject having a disease refers to subjecting the subject to a pharmaceutical treatment, e.g., the administration of a drug, such that the extent of the disease is decreased or prevented. For example, treating results in the reduction of at least one sign or symptom of the disease or condition. Treatment includes (but is not limited to) administration of a composition, such as a pharmaceutical composition, and may be performed either prophylactically, or subsequent to the initiation of a pathologic event. Treatment can require administration of an agent and/or treatment more than once.

Methods for Diagnosing CIPN

In a first aspect, the invention relates to a method of diagnosing CIPN in a patient treated by chemotherapy, said method comprising the step of assessing the sudomotor function of patient. The inventors have surprisingly found that sudomotor activity is altered in subjects affected by CIPN. Sudomotor function is significantly associated with symptoms of CIPN in subjects treated by chemotherapy. Thus, measuring sudomotor function provides a quick and simple, non-invasive test for diagnosing CIPN in a subject, which is particularly advantageous. In particular, no clinical test is required and, thus, the presence of neurologist is not necessary. Moreover, the method of the invention enables an early diagnosis of CIPN before the onset of symptoms, which cannot be done using the current TSNc test.

Sweat glands are innervated by thin and unmyelinated sympathetic C fibers. The inventors have found that, when these fibers are compromised, as in e.g. subjects affected by CIPN, the resulting bioelectrical potential in the CIPN sweat gland is altered. Thus sweat chloride movements upon electric stimulation, and therefore the electrochemical skin conductance, are impaired in subjects suffering from CIPN. This is the basis of method of the invention, which explores the sweat duct using active electrophysiology, such as measurement of electrochemical skin conductance after application of a low direct voltage via stainless steel electrodes.

This low voltage applied on human skin generates a current through reverse iontophoresis, i.e. ion movements via sweat duct pores in response to electric stimulation and local electrochemical reactions between those ions and stainless steel electrodes. On human skin, at the voltages applied in the current invention (i.e. below 10 V), ions cannot go through the stratum corneum because of the very high electrical capacity of its dense lipid layers. Thus, the only way for ions in human skin to move in such an electric field is via sweat duct pores, and the measurement of electrochemical skin conductance as carried out in the present invention is correlated to the sweat chloride movements impaired by CIPN, and is as such a good tool for its diagnosis.

In a first embodiment, the method of the invention comprises a step of assessing the sudomotor function of said patient, wherein said step involves measuring the electrochemical skin conductance of the patient. According to this embodiment, the invention provides a method of diagnosing CIPN in a cancer patient treated by chemotherapy, said method comprising the step of:
a) measuring the electrochemical skin conductance of the patient; and
b) determining whether said patient suffers from CIPN based on the measurement of step a).

Description of a Diagnosis System According to the Invention

The inventors have developed a specific diagnosis system for measuring the electrochemical skin conductance of a subject. This system comprises an anode and a cathode, intended to be placed on different regions of the subject body, and an adjustable DC source, which is controlled in order to feed the anode with a DC current. Thus in another embodiment, the method of the invention comprises a step of assessing the sudomotor function of patient, wherein said step involves determining the electrochemical skin conductance of the patient with a system comprising an anode and a cathode, intended to be placed on different regions of the subject body, and an adjustable DC source, which is controlled in order to feed the anode with a DC current.

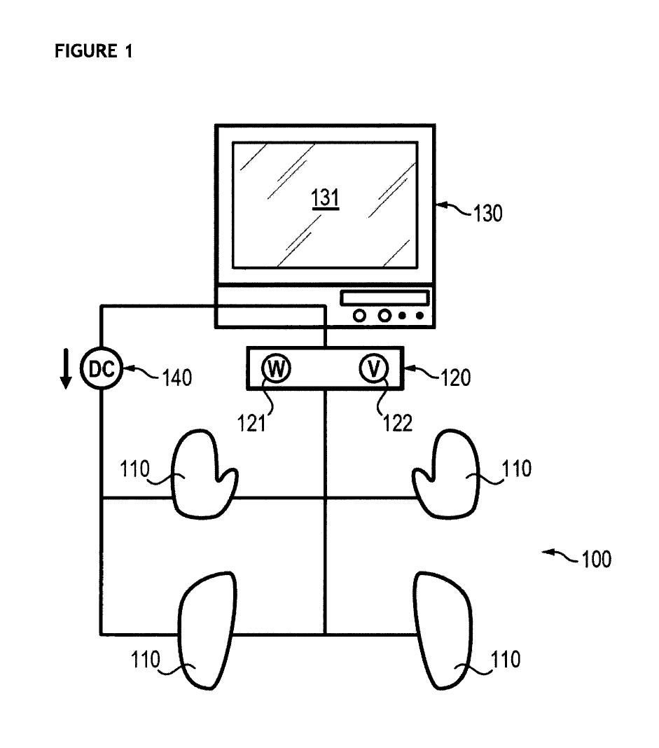

Such a system 100 for diagnosing a patient by assessing electrochemical skin conductance is shown in FIG. 1. The system 100 comprises a series of large area electrodes 110, preferably four electrodes 110, on which the patient can place his hands and feet. The sites of the electrodes 110 have been chosen because of their high density of sweat glands.

The electrodes 110 can be made of stainless steel. Their individual surface area is comprised between 50 cm² and 200 cm², so that they cover substantially all the surface of the hand palms and of the feet soles. Yet they can be adapted for children or even infants.

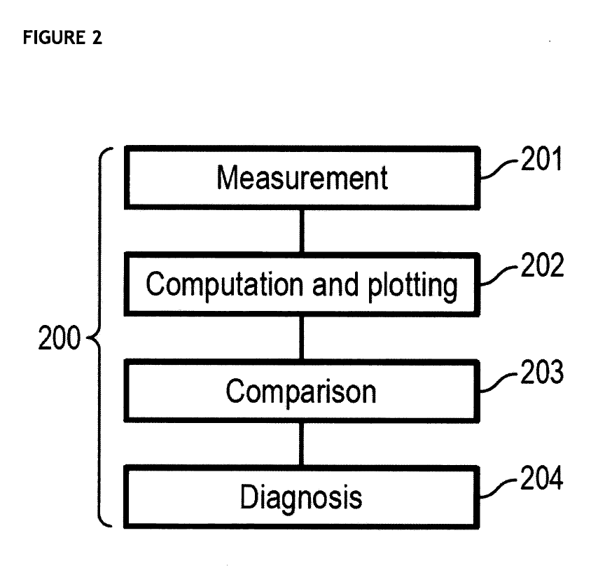

The electrodes are connected to an electronic board 120 managed by a computer 130 for collecting, computing, and storing data. They are also connected to an adjustable DC source 140, which is controlled by an operator or the electronic board to feed the electrodes 110 with a DC current of a determined voltage. The electronic board 120 is also designed to measure the voltage potential of each electrode through a voltmeter 121, as well as the current between two electrodes through a Wheatstone bridge 122. The diagnosing system can also be equipped with a display 131, designed for displaying the measured data as well as the results of the computations carried out on said data. The diagnosis method 200 according to the invention will now be described with reference to FIG. 2.

Measurement Step 201

In order to diagnose CIPN in a patient, said patient places his hands and feet on the large area stainless steel electrodes 110, and stands up without moving his hands and feet during the 2 minutes that lasts the measurement. The measurement 201 is carried out independently for the two feet and for the two hands. For one measurement configuration (for example right-hand, left-hand), one electrode is used as an anode, and the other one is used as a cathode.

The anode is then fed with DC current. The anode is applied an initial voltage, comprised between 0.5 V and 1.5 V, preferably equal or close to 1 V, during a duration comprised between 0.5 second and 2 seconds, preferably 1 second. The duration must last long enough to allow the stabilization of electrochemical phenomena in the body, in the vicinity of the electrodes. The applied current induces voltage on the cathode, and a current going through the body towards the cathode, as previously explained.

Both electrodes voltages and current through them are measured and stored by the electronic board 120 at measurement step 201. Then, the voltage applied on the anode is increased by a step comprised between 0.1 V and 0.3 V, preferably 0.2 V. For instance, the voltage applied is increased from 1 V up to 1.2 V. This voltage value is applied on the anode during between 0.5 second and 2 seconds, preferably 1 second, and a new measurement is performed. Such a progressive step by step increase of between 0.1 V and 0.3 V, preferably 0.2 V, during preferably 1 second is applied until a maximal voltage below 10V, preferably of between 3.5 V and 4 V, and even more preferably of 3.8 V is reached.

This stepwise increase represents preferably a total of 15 measurements, when the minimum voltage value is 1 V, the maximum voltage value is 3.8 V, and the step is 0.2 V. The following results have been obtained with these experiments conditions. The same series of measurements can also be carried out in reverse, by applying successive pulses of decreasing voltages. The same series of measurements can then be carried out with the electrodes being reversed (anode becoming cathode and vice-versa), and the same can be carried out on the feet.

Computation and Plotting 202

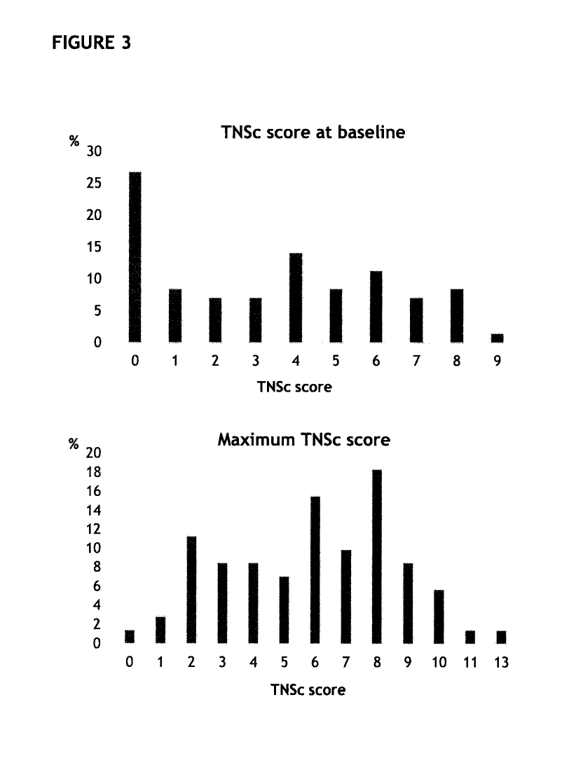

Once the electrodes potentials have been recorded, the electronic board computes the difference in voltages between the anode and the cathode, noted Δ(Anode−Cathode), for each DC voltage applied to the anode, as illustrated in FIG. 3. The current measured at each voltage is then plotted against the difference in voltages Δ(Anode−Cathode). The curve obtained is linear when voltage applied to the anode is low. The electrochemical skin conductance, being the slope of the curve, i.e. the ratio between the current measured and the difference in voltages Δ(Anode−Cathode), is then computed. This step of computation and plotting is referenced as 202 on FIG. 2.

Comparison with Reference Value

In an embodiment of the method of the invention, determining whether said patient suffers from CIPN involves comparing the electrochemical skin conductance of the patient to a reference value (203). According to this embodiment, the invention relates to a method of diagnosing CIPN in a cancer patient treated by chemotherapy, said method comprising the steps of:
a) measuring the electrochemical skin conductance of the patient;
b) comparing the measurement of step a) with a reference value; and
c) determining whether said patient suffers from CIPN based on the comparison of step b).

The reference value corresponds for example to the value of said conductance in a healthy subject. Comparison of the skin conductance of said patient with a healthy patient leads to the formulation of a diagnosis of CIPN at a specific moment of the chemotherapy treatment of said patient. According to this embodiment, the determination of step b) of the method of the invention comprises comparing the value of the electrochemical skin conductance of the patient with the value of the electrochemical skin conductance of a healthy subject.

According to the present invention, a decreased value of said conductance in said patient relative to said healthy subject is indicative of a diagnosis of CIPN. On the other hand, an increased or a stable value of said conductance is indicative of the absence of CIPN in said patient.

The reference value may also be a value of said conductance in the same patient at an earlier time point. For example, the reference value may be the value of the electrochemical skin conductance of the same patient, before the start of the chemotherapy treatment.

According to this embodiment, the determination of step b) of the method of the invention comprises comparing the value of the electrochemical skin conductance of the patient at a first point during chemotherapy treatment with the value of the electrochemical skin conductance of said patient before chemotherapy. In other words, this embodiment provides a diagnosis of CIPN with no interference from interindividual variations in sudomotor function and, in particular, in electrochemical skin conductance. According to the present invention, a decreased value of said conductance in said patient during chemotherapy relative to before chemotherapy is indicative of a diagnosis of CIPN. On the other hand, an increased or a stable value of said conductance is indicative of the absence of CIPN.

Methods for Monitoring CIPN

The method of the invention is particularly advantageous, since subjects suffering from CIPN can detected before they even develop clinical symptoms. Therefore it is possible to determine at a very early stage if the patient receiving a chemotherapy treatment is at risk of developing CIPN.

This represents an important and medically useful discovery. This discovery enables the discrimination of patients, at the beginning of a chemotherapy treatment, in a group of patients who are likely not to develop CIPN and a group of patients likely to develop CIPN. Determining at a very early stage that a patient is likely to develop CIPN may help adapting the treatment of said patient in order to minimize the severity of the CIPN symptoms of said patient. This diagnostic tool may also assist physicians in identifying patients who are likely not to develop CIPN and could thus receive higher doses of chemotherapy with minimal side effects.

In another aspect, the invention also relates to a method for determining the likelihood that a patient treated by chemotherapy will develop symptoms of CIPN, said method comprising the steps of:
a) assessing the sudomotor function of said patient; and
b) determining the likelihood that said patient will develop symptoms of CIPN based on the assessment of step a).

In a preferred embodiment, the assessment of the sudomotor function of said patient involves measuring the electrochemical skin conductance of the patient. According to this embodiment, the invention also relates to a method for determining the likelihood that a patient treated by chemotherapy will develop symptoms of CIPN, said method comprising the steps of:
a) measuring the electrochemical skin conductance of said patient; and
b) determining the likelihood that said patient will develop symptoms of CIPN based on the assessment of step a).

In a preferred embodiment, the determination of step b) comprises comparing the measurement of step a) with a reference. According to this embodiment, the invention relates to a method of determining the likelihood that a patient treated by chemotherapy will develop symptoms of CIPN, said method comprising the steps of:
a) measuring the electrochemical skin conductance of the patient;
b) comparing the measurement of step a) with a reference value; and
c) determining the likelihood that said patient will develop symptoms of CIPN based on the comparison of step b).

The reference value corresponds for example to the value of said conductance in a healthy subject. According to this embodiment, the determination of step b) of the method of the invention comprises comparing the value of the electrochemical skin conductance of the patient with the value of the electrochemical skin conductance of a healthy subject. According to the present invention, a decreased value of said conductance in said patient relative to said healthy subject indicates that said patient is likely to develop CIPN symptoms. On the other hand, an increased or a stable value of said conductance is indicative of the low likelihood of development of CIPN symptoms in said patient.

The reference value may also be a value of said conductance in the same patient at an earlier time point. For example, the reference value may be the value of the electrochemical skin conductance of the same patient, before the start of the chemotherapy treatment. According to this embodiment, the determination of step b) of the method of the invention comprises comparing the value of the electrochemical skin conductance of the patient at a first point during chemotherapy treatment with the value of the electrochemical skin conductance of said patient before chemotherapy.

According to the present invention, a decreased value of said conductance in said patient during chemotherapy relative to before chemotherapy indicates that it is likely that the patient will develop CIPN symptoms. On the other hand, an increased or a stable value of said conductance indicates that it is not likely that the patient will develop CIPN symptoms.

The invention is also particularly useful because it enables to track the progression of CIPN based on objective, reliable data. CIPN severity is generally assessed using the TNSc scales, although significant inter-observer disagreement may exist using these scales. Moreover, TNSc uses only clinical measures, including impairment and disability measures, which could lead to misinterpretation of the results and unpredictable under- or overestimation of the effect. This uncertainty may lead to different interpretations of the results of the same clinical situation by different clinicians. It can thus be difficult for the clinician to detect subtle changes in the evolution of the condition of a patient.

By comparison, the invention provides a reliable tool for monitoring the progression of CIPN in a patient. Assessment of the sudomotor function, and in particular measurement of the electrochemical skin conductance, is easily obtained. The measurement of sudomotor function provides an objective assessment of the likelihood that the patient will develop CIPN symptoms. In addition, the intrinsic quantitative nature of said measurement affords an easy and reliable way of comparing progression of CIPN in the patient with a reference, e.g., the general population or said patient at a different time point without difficulty. Such a comparison offers a quick and reliable image of the progression of CIPN in a patient, before any detectable change of the clinical symptoms.

In another aspect, the invention also relates to a method for monitoring the progression of CIPN in a patient. In an embodiment, the invention relates to a method of prognosing CIPN in a patient, said method comprising the steps of:
a) assessing the sudomotor function of said patient at first time point;
b) assessing the sudomotor function of said patient at second time point; and
c) comparing the assessment of step a) and the assessment of step b);
d) prognosing CIPN in said patient based on the comparison of step c).

In a preferred embodiment, the assessment of the sudomotor function of said patient involves measuring the electrochemical skin conductance of the patient. According to this embodiment, the invention also relates to a method of prognosing CIPN in a patient, said method comprising the steps of:
a) measuring the electrochemical skin conductance of said patient at first time point;
b) measuring the electrochemical skin conductance of said patient at first time point; and
c) comparing the measurement of step a) and the measurement of step b);
d) prognosing CIPN in said patient based on the comparison of step c).

The prognosis in step d) of the method of the invention may be positive if e.g., the measurement of step b) is superior or equal to the measurement of step a). Alternatively, the prognosis in step d) of the method of the invention may be of a worsening of said CIPN, if e.g., the sudomotor function decreases between steps a) and b).

Methods for Adapting Chemotherapy Treatments

A subject identified as being likely to develop CIPN symptoms would benefit from an adaptation of his/her chemotherapy treatment before the appearance of the CIPN symptoms. A practitioner treats proliferative diseases such as cancer by taking actions to ameliorate the causes or symptoms of the disease in a patient. Treatment of cancer comprises administering therapy to a patient. Therapy may include: selecting and administering one or more chemotherapeutic drugs to the patient, adjusting the dosage of the chemotherapeutic drugs, adjusting the dosing schedule of the drug, and adjusting the length of the therapy. Chemotherapeutic agents are selected by practitioners based on the nature of the cancer, the patient's response to the cancer and the patient's response to the agent. The dosage of the chemotherapeutic agents can be adjusted as well by the practitioner based on the nature of the drug, the nature of the cancer, the patient's response to the cancer, and the patient's response to the drug. The dosing schedule can also be adjusted by the practitioner based on the nature of the drug, the nature of the cancer, the patient's response to the cancer, and the patient's response to the drug. Also, the length of the therapy can be adjusted by the practitioner based on the nature of the chemotherapeutic agents, the nature of the cancer, the patient's response to the cancer, the patient's response to the drug. Also, the practitioner can select between a single drug therapy, a dual drug therapy, i.e. between administering a single chemotherapeutic agent and administering a combination of two or more chemotherapeutic agents.

Thus, it is also an aspect of the invention to provide a method for adapting a chemotherapy treatment in a patient, wherein said method comprises the steps of:
a) assessing the sudomotor function of said patient;
b) determining the likelihood that said patient will develop symptoms of CIPN based on the assessment of step a); and
c) adapting the chemotherapy treatment of said patient based on the likelihood of step b).

In a preferred embodiment, the sudomotor function of said patient is assessed by measuring the electrochemical skin conductance of the patient. In another preferred embodiment, the sudomotor function of said patient is compared to a reference value. According to this embodiment, the determination of step b) of the method of the invention comprises comparing the value of the electrochemical skin conductance of the patient with the value of the electrochemical skin conductance of a healthy subject.

According to the present invention, a decreased value of said conductance in said patient relative to said healthy subject indicates that the patient is likely to develop symptoms of CIPN. On the other hand, an increased or a stable value of said conductance indicates that the patient is not likely to develop symptoms of CIPN. The reference value may also be a value of said conductance in the same patient at an earlier time point. For example, the reference value may be the value of the electrochemical skin conductance of the same patient, before the beginning of the chemotherapy treatment.

According to this embodiment, the determination of step b) of the method of the invention comprises comparing the value of the electrochemical skin conductance of the patient at a first point with the value of the electrochemical skin conductance of said patient before the treatment. In other words, this embodiment provides an indication of the evolution of the activity of the autonomous system with no interference from interindividual variations in sudomotor function and, in particular, in electrochemical skin conductance.

According to the present invention, an increased or a stable value of said conductance in said patient during the chemotherapy treatment relative to before the beginning of the treatment is indicative of a recovery of the activity of the C fibers, meaning that the patient is not likely to develop symptoms of CIPN. On the other hand, a decreased value of said conductance is indicative of the absence of such a recovery. In this case, it is likely that the patient will develop symptoms of CIPN.

Said adaptation of the chemotherapy therapy may consist in:
a reduction or suppression of said chemotherapy if the patient is assessed as being likely to develop symptoms of CIPN, or
the continuation or an augmentation of said chemotherapy if the patient is assessed as not being likely to develop symptoms of CIPN.

In one embodiment, the practitioner adjusts the therapy based on the patient's level of electrochemical skin conductance compared to a reference level. In one embodiment, the practitioner adjusts the therapy by selecting and administering a different drug. In one embodiment, the practitioner adjusts the therapy by selecting and administering a different combination of drugs. In one embodiment, the practitioner adjusts the therapy by adjusting drug dosage. In one embodiment, the practitioner adjusts the therapy by adjusting dose schedule. In one embodiment, the practitioner adjusts the therapy by adjusting length of therapy. In one embodiment, the practitioner adjusts the therapy by selecting and administering a different drug combination and adjusting drug dosage. In one embodiment, the practitioner adjusts the therapy by selecting and administering a different drug combination and adjusting dose schedule. In one embodiment, the practitioner adjusts the therapy by selecting and administering a different drug combination and adjusting length of therapy. In one embodiment, the practitioner adjusts the therapy by adjusting drug dosage and dose schedule. In one embodiment, the practitioner adjusts the therapy by adjusting drug dosage and adjusting length of therapy. In one embodiment, the practitioner adjusts the therapy by adjusting dose schedule and adjusting length of therapy. In one embodiment, the practitioner adjusts the therapy by selecting and administering a different drug, adjusting drug dosage, and adjusting dose schedule. In one embodiment, the practitioner adjusts the therapy by selecting and administering a different drug, adjusting drug dosage, and adjusting length of therapy. In one embodiment, the practitioner adjusts the therapy by selecting and administering a different drug, adjusting dose schedule, and adjusting length of therapy. In one embodiment, the practitioner adjusts the therapy by adjusting drug dosage, adjusting dose schedule, and adjusting length of therapy. In one embodiment, the practitioner adjusts the therapy by selecting and administering a different drug, adjusting drug dosage, adjusting dose schedule, and adjusting length of therapy.

In one embodiment where there is a decreased level of level of electrochemical skin conductance with respect to a reference level, treatment comprises a less aggressive therapy than a reference therapy. In one embodiment a less aggressive therapy comprises not administering drugs and taking a “watchful waiting” approach. In one embodiment a less aggressive therapy comprises delaying administration of chemotherapeutic agents. In one embodiment a less aggressive therapy comprises selecting and administering less potent drugs. In one embodiment a less aggressive therapy comprises decreasing dosage of chemotherapeutic agents. In one embodiment a less aggressive therapy comprises decreasing the frequency of the dose schedule. In one embodiment a less aggressive therapy comprises shortening length of therapy. In one embodiment, less aggressive therapy comprises selecting and administering less potent drugs and decreasing drug dosage. In one embodiment, less aggressive therapy comprises selecting and administering less potent drugs and decreasing dose schedule. In one embodiment, less aggressive therapy comprises selecting and administering less potent drugs and shortening length of therapy. In one embodiment, less aggressive therapy comprises decreasing drug dosage and decreasing dose schedule. In one embodiment, less aggressive therapy comprises decreasing drug dosage and shortening length of therapy. In one embodiment, less aggressive therapy comprises decreasing dose schedule and shortening length of therapy. In one embodiment, less aggressive therapy comprises selecting and administering less potent drugs, decreasing drug dosage, and decreasing dose schedule. In one embodiment, less aggressive therapy comprises selecting and administering less potent drugs, decreasing drug dosage, and shortening length of therapy. In one embodiment, less aggressive therapy comprises selecting and administering less potent drugs, decreasing dose schedule, and shortening length of therapy. In one embodiment, less aggressive therapy comprises decreasing drug dosage, decreasing dose schedule, and shortening length of therapy. In one embodiment, less aggressive therapy comprises selecting and administering less potent drugs, decreasing drug dosage, decreasing dose schedule, and shortening length of therapy.

In one embodiment a less aggressive therapy comprises selecting and administering a single therapy instead of a dual therapy, i.e. a single chemotherapeutic agent rather than a combination of two chemotherapeutic agents. In one embodiment a less aggressive therapy comprises delaying administration of chemotherapeutic agents and selecting and administering a single therapy instead of a dual therapy. In one embodiment a less aggressive therapy comprises selecting and administering a single therapy instead of a dual therapy and selecting and administering less potent drugs. In one embodiment a less aggressive therapy comprises selecting and administering a single therapy instead of a dual therapy and decreasing dosage of chemotherapeutic agents. In one embodiment a less aggressive therapy comprises selecting and administering a single therapy instead of a dual therapy and decreasing the frequency of the dose schedule. In one embodiment a less aggressive therapy comprises selecting and administering a single therapy instead of a dual therapy and shortening length of therapy. In one embodiment, less aggressive therapy comprises selecting and administering a single therapy instead of a dual therapy and selecting and administering less potent drugs and decreasing drug dosage. In one embodiment, less aggressive therapy comprises selecting and administering a single therapy instead of a dual therapy and selecting and administering less potent drugs and decreasing dose schedule. In one embodiment, less aggressive therapy comprises selecting and administering a single therapy instead of a dual therapy, selecting and administering less potent drugs and shortening length of therapy. In one embodiment, less aggressive therapy comprises selecting and administering a single therapy instead of a dual therapy, decreasing drug dosage and decreasing dose schedule. In one embodiment, less aggressive therapy comprises selecting and administering a single therapy instead of a dual therapy, decreasing drug dosage and shortening length of therapy. In one embodiment, less aggressive therapy comprises selecting and administering a single therapy instead of a dual therapy, decreasing dose schedule and shortening length of therapy. In one embodiment, less aggressive therapy comprises selecting and administering a single therapy instead of a dual therapy, selecting and administering less potent drugs, decreasing drug dosage, and decreasing dose schedule. In one embodiment, less aggressive therapy comprises selecting and administering a single therapy instead of a dual therapy, selecting and administering less potent drugs, decreasing drug dosage, and shortening length of therapy. In one embodiment, less aggressive therapy comprises selecting and administering a single therapy instead of a dual therapy, selecting and administering less potent drugs, decreasing dose schedule, and shortening length of therapy. In one embodiment, less aggressive therapy comprises selecting and administering a single therapy instead of a dual therapy, decreasing drug dosage, decreasing dose schedule, and shortening length of therapy. In one embodiment, less aggressive therapy comprises selecting and administering a single therapy instead of a dual therapy, selecting and administering less potent drugs, decreasing drug dosage, decreasing dose schedule, and shortening length of therapy. In one embodiment a less aggressive therapy comprises selecting and administering a single therapy instead of a dual therapy.

Chemotherapy-Treated Subjects

According to the present invention, the subject at risk of developing CIPN is evaluated is treated with chemotherapy. Chemotherapy involves the administration of a chemotherapeutic agent. Chemotherapeutic agent or agents may include, for example, antimetabolites (i.e., folate antagonists, purine antagonists, and pyrimidine antagonists), bleomycins, DNA alkylating agents (i.e., nitrosoureas, cross linking agents, and alkyating agents), hormones, aromatase inhibitors, monoclonal antibodies, antibiotics, platinum complexes, protesome inhibitors, taxane analogs, vinca alkaloids, topoisomerase inhibitors (i.e., anthracyclines, camptothecins, podophyllotoxins), tyrosine kinase inhibitors, or a combination thereof.

In another embodiment, chemotherapeutic agents may include, for example, a platinum complex, a vinca analog, a taxane analog, an alkylating agent, an antimetabolite, a proteasome inhibitor, or a combination thereof. Platinum complexes may include, for example, cisplatin, oxaliplatin, eptaplatin, lobaplatin, nedaplatin, carboplatin, satraplatin, picoplatin and the like. Vinca alkaloids may include, for example, vincristine, vinblastine, vinorelbine, vindesine, and the like. Taxanes may include, for example, paclitaxel, docetaxel, and various formulations and analogs thereof.

Alkylating agents may include, for example, dacarbazine, procarbazine, temozolamide, thiotepa, mechlorethamine, chlorambucil, L-phenylalanine mustard, melphalan, ifosphamide, cyclophosphamide, mefosphamide, perfosfamide, trophosphamide, busulfan, carmustine, lomustine, thiotepa, semustine, and the like. Antimetabolites include pemetrexed disodium, 5 azacitidine, capecitabine, carmofur, cladribine, clofarabine, cytarabine, cytarabine ocfosfate, cytosine arabinoside, decitabine, deferoxamine, doxifluridine, eflornithine, enocitabine, ethnylcytidine, fludarabine, 5 fluorouracil alone or in combination with leucovorin, gemcitabine, hydroxyurea, melphalan, mercaptopurine, 6 mercaptopurine riboside, methotrexate, mycophenolic acid, nelarabine, nolatrexed, ocfosfate, pelitrexol, pentostatin, raltitrexed, Ribavirin, triapine, tnmetrexate, S-1, tiazofurin, tegafur, TS-1, vidarabine, UFT and the like. Proteasome inhibitors may include, for example, bortezomib. Topoisomerase inhibitors include aclarubicin, 9-aminocamptothecin, amonafide, amsacrine, becatecarin, belotecan, irinotecan hydrochloride, camptothecin, dexrazoxine, diflomotecan, edotecarin, epirubicin, etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

In another embodiment, chemotherapeutic agents are bortezomib, carboplatin, cisplatin, gemcitabine, misonidazole, oxaliplatin, procarbazine, thalidomide, docetaxel, hexamethylmelamine, paclitaxel, vincristine, vinblastine, or vinorelbine. In one embodiment of the invention, the chemotherapeutic agent is carboplatin and the compound of formula (I) is 2-[(2R)-2-methylpyrrolidin-2-yl]-IH-benzimidazole-4-carboxamide. Yet another embodiment further comprises the chemotherapeutic agent topotecan. Yet another embodiment further comprises the chemotherapeutic agent cyclophosphamide. In one embodiment of the invention, the chemotherapeutic agent is cisplatin and the compound of formula (I) is 2-[(2R)-2-methylpyrrolidin-2-yl]-IH-benzimidazole-4-carboxamide. Yet another embodiment further comprises the chemotherapeutic agent cyclophosphamide.

In one embodiment of the invention, the chemotherapeutic agent is oxaliplatin and the compound of formula (I) is 2-[(2R)-2-methylpyrrolidin-2-yl]-IH-benzimidazole-4-carboxamide. Yet another embodiment further comprises the chemotherapeutic agent capecitabine. Yet another embodiment further comprises the chemotherapeutic agents 5-fluorouracil and leucovorin.

In one embodiment of the invention, the chemotherapeutic agent is paclitaxel and the compound of formula (I) is 2-[(2R)-2-methylpyrrolidin-2-yl]-IH-benzimidazole-4-carboxamide. Yet another embodiment further comprises the chemotherapeutic agent cisplatin. Yet another embodiment further comprises the chemotherapeutic agents doxorubicin and cyclophosphamide.

In one embodiment of the invention, the chemotherapeutic agent is docetaxel and the compound of formula (I) is 2-[(2R)-2-methylpyrrolidin-2-yl]-IH-benzimidazole-4-carboxamide. Yet another embodiment further comprises the chemotherapeutic agents doxorubicin and cyclophosphamide. Yet another embodiment further comprises the chemotherapeutic agents cisplatin and fluorouracil. In one embodiment of the invention, the chemotherapeutic agent is vinorelbine and the compound of formula (I) is 2-[(2R)-2-methylpyrrolidin-2-yl]-IH-benzimidazole-4-carboxamide. Yet another embodiment further comprises the chemotherapeutic agent cisplatin.

In one embodiment of the invention, the chemotherapeutic agents are carboplatin and docetaxel and the compound of formula (I) is 2-[(2R)-2-methylpyrrolidin-2-yl]-IH-benzimidazole-4-carboxamide. In one embodiment of the invention, the chemotherapeutic agents are cisplatin and docetaxel and the compound of formula (I) is 2-[(2R)-2-methylpyrrolidin-2-yl]-IH-benzimidazole-4-carboxamide. In one embodiment of the invention, the chemotherapeutic agents are carboplatin and paclitaxel and the compound of formula (I) is 2-[(2R)-2-methylpyrrolidin-2-yl]IH-benzimidazole-4-carboxamide. Yet another embodiment further comprises the chemotherapeutic agent bevacizumab.

In one embodiment of the invention, the chemotherapeutic agents are cisplatin and paclitaxel and the compound of formula (I) is 2-[(2R)-2-methylpyrrolidin-2-yl]-IH-benzimidazole-4-carboxamide. In one embodiment of the invention, the chemotherapeutic agents are carboplatin and gemcitabine and the compound of formula (I) is 2-[(2R)-2-methylpyrrolidin-2-yl]-IH-benzimidazole-4-carboxamide. In one embodiment of the invention, the chemotherapeutic agents are cisplatin and gemcitabine and the compound of formula (I) is 2-[(2R)-2-methylpyrrolidin-2-yl]-IH-benzimidazole-4-carboxamide. In one embodiment of the invention, the chemotherapeutic agents are cisplatin and vinorelbine and the compound of formula (I) is 2-[(2R)-2-methylpyrrolidin-2-yl]-IH-benzimidazole-4-carboxamide.

In another embodiment, the chemotherapeutic agent or agents is administered for the treatment of cancer. In one embodiment of the invention, the cancer being treated is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. In yet another embodiment of the invention, the cancer being treated is selected from the group consisting of ovarian cancer, cervical cancer, colorectal cancer, prostate cancer, breast cancer, gastric adenocarcinoma, head and neck cancer, testicular cancer, leukemia, neuroblastoma, Hodgkin's lymphoma, non-Hodgkin's lymphoma, and non-small cell lung cancer.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting. Other features and advantages of the invention will be apparent from the following detailed description, and from the claims.

### EXAMPLES

This study aimed at evaluating the SUDOSCAN® device (from Impeto Medical, Paris, France) in the detection and follow-up of CIPN in patients treated with neurotoxic chemotherapy even before occurrence of symptoms.

Material and Methods

### Inclusion Criteria

Adults patients:
Both male and female;
   Aged between 18 and 70 years;
   Receiving a potentially neurotoxic chemotherapy: vinca alkaloids, platinum salts or taxans;
   Having given their informed consent.
      The population characteristics are shown in Table 1.

TABLE 1
Characteristics of the patient population of the study

| | Total population (N = 71) |
|---|---|
| Age (years) | 62.0 ± 13.1 |
| Gender | |
| Female | 22 (31.0) |
| Male | 49 (69.0) |
| Height (cm) | 172.2 ± 7.7 |
| Weight (kg) | 70.1 ± 14.1 |
| Tumor location | |
| Bronchi/Oropharynx | 14 (19.7) |
| Colon | 13 (18.3) |
| Digestive organs | 14 (19.7) |
| Other | 30 (42.3) |
| Treatment | |
| Carboplatin | 10 (14.1) |
| Oxaliplatin | 28 (39.4) |
| Paclitaxel | 19 (26.8) |
| Other | 18 (25.4) |

Non Inclusion Criteria
Previous treatment by vinca alkaloids, platiunum salts or taxans;
Pre-existing neuropathy;
Diabetes;
Implanted electrical device (cardiac stimulator; defibrillator);
Limb amputation
Current neurotoxic treatment.

Protocol

At each chemotherapy infusion, accumulated dose of chemotherapy was calculated and the Total Neuropathy Score (TNSc) was performed. To measure small fiber neuropathy, patients were assessed by the SUDOSCAN® device (3 minutes exam). The device measures the Electrochemical Skin Conductance (ESC) of the hands and feet expressed in microSiemens (μS).

Results

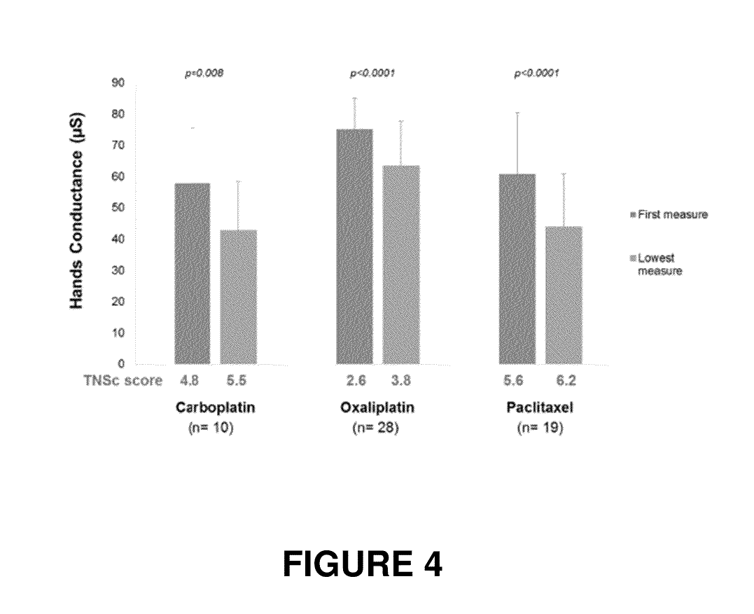

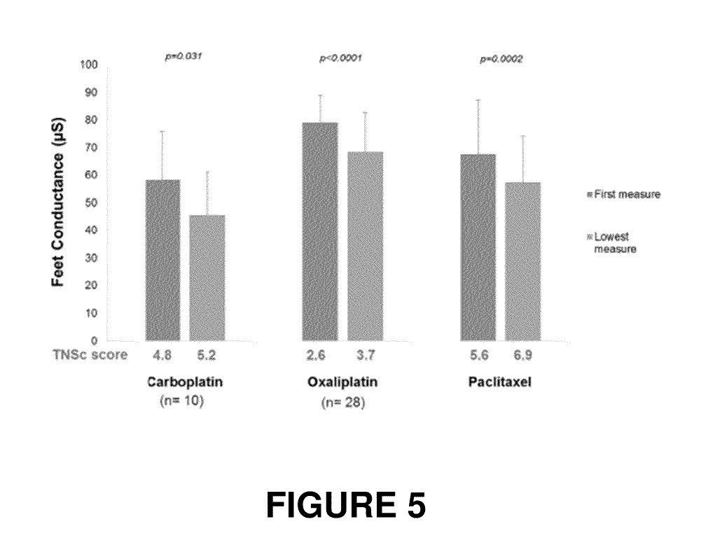

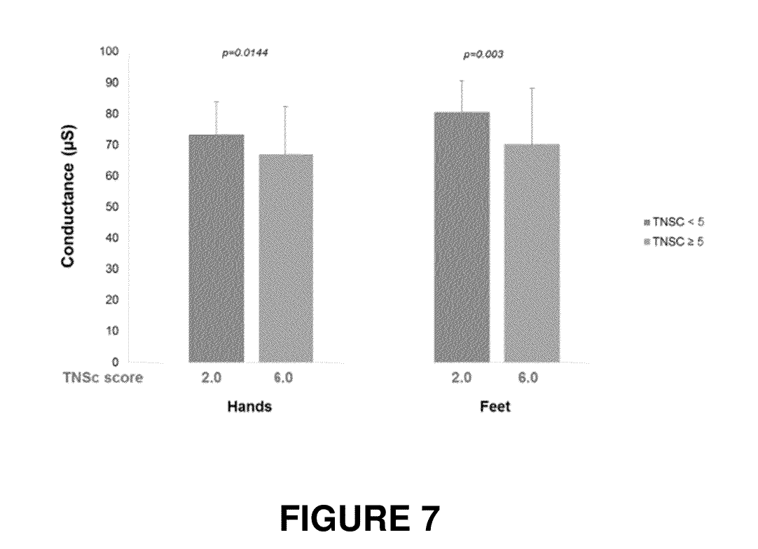

Results displayed in FIGS. 4 and 5 show that a decrease in hands and feet conductance is observed after initiation of the chemotherapy for all three treatment received. This is confirmed by the concomitant increase in TNSc. Indeed, feet conductance and TNSc were negatively correlated in patients receiving oxaliplatin (r=−0.4; p<0.0001; FIG. 7), demonstrating that small fiber neuropathy can be followed through measurement of electrochemical skin conductance with the SUDOSCAN® device. A decrease in the mean hands and feet conductance could be detected after the start of the treatment even in asymptomatic patients, i.e. in patients with a null score for the first and second questions of the TNSc. This indicates that the SUDOSCAN® device could help detect and quantify CIPN even in asymptomatic patients.

1. A method of diagnosing a CIPN in a cancer patient treated by chemotherapy, the method comprising:
a) measuring electrochemical skin conductance of the patient; and
b) determining if the patient suffers from the CIPN based on the measurement of step a).

2. The method of claim 1, wherein the electrochemical skin conductance is determined with a system comprising an anode and a cathode, placed on different regions of the patient, and an adjustable DC source, and the method further comprises controlling the DC source to feed the anode with a DC current.

3. The method of claim 2, wherein step a) comprises:
applying DC voltage pulses of varying voltage values to the anode for given durations allowing the stabilization of electrochemical phenomena in the body in the vicinity of the electrodes;
collecting data representative of the current between the anode and the cathode, and of the potentials of the anode and the cathode, for the different DC voltages; and
from the data, computing data representative of the electrochemical skin conductance of the patient.

4. A method according to claim 1, wherein the electrochemical skin conductance value at a given voltage applied on the anode is determined as the ratio between the current through the anode and the cathode and the voltage difference between the anode and the cathode.

5. A method according to claim 1, wherein a duration of DC voltage applied on an anode for each step before a voltage change is between 0.5 and 2 seconds.

6. A method according to claim 1, wherein voltage values increase and/or decrease stepwise.

7. A method according to claim 6, wherein the step increase or decrease between two successive pulses of the voltage valves is between 0.1 and 0.3 V.

8. A method according to claim 7, wherein the voltage values are in the range from about 0.5 V and 10 V.

9. A method according to claim 1, wherein computed data relative to the electrochemical skin conductance values of the patient include the electrochemical skin conductance value of the patient as a function of a voltage value applied to an anode.

10. A method according to claim 1, wherein computed data relative to the electrochemical skin conductance values of the patient include the electrochemical skin conductance value of the patient at a voltage value between about 1.4 and 1.8 V.

11. A method according to claim 1, wherein computed data relative to the electrochemical skin conductance values of the patient also includes the electrochemical skin conductance value of the patient at a voltage between about 3.4 V and 3.8 V.

12. A method according to claim 9, wherein the computed data relative to the electrochemical skin conductance values of the patient include the difference and/or the ratio between two electrochemical skin conductance values of the patient for two different voltage values applied to the anode.

13. A method according to claim 11, wherein the computed data relative to the electrochemical skin conductance values of the patient include the difference and/or the ratio between two electrochemical skin conductance values of the patient for an intermediate and a high voltage value applied to the anode.

14. A method according to claim 12, further comprising a reconciling step comprises determining whether the difference and/or the ratio between two electrochemical skin conductance values of the patient for intermediate and high voltage values applied to the anode is below a given threshold.

15. A method according to claim 14, wherein the intermediate voltage is comprised between 1.4 V and 1.8 V, and the high voltage value is between 3.4 V and 3.8 V.

16. A method according to claim 1, further comprising using hands and feet electrodes for the measuring step.

17. A method according to claim 16, wherein the electrodes cover substantially all the surface of the hand palms and of the feet soles.

18. The method of claim 1, wherein step b) comprises comparing the electrochemical skin conductance of the patient with a reference value.

19. The method of claim 18, wherein the reference value is the electrochemical skin conductance of a healthy individual.

20. The method of claim 18, wherein the reference value is the electrochemical skin conductance of the patient at a second time point.

21. The method of claim 1, wherein the cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carincoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

22. The method of claim 1, wherein the chemotherapy treatment comprises administering of at least one chemotherapeutic agent selected from the group of: antimetabolites, bleomycins, DNA alkylating agents, hormones, aromatase inhibitors, monoclonal antibodies, antibiotics, platinum complexes, protesome inhibitors, taxane analogs, vinca alkaloids, topoisomerase inhibitors, and tyrosine kinase inhibitors.

23. A method for determining the likelihood that a cancer patient treated by chemotherapy will develop symptoms of CIPN, the method comprising:
a) assessing a sudomotor function of a patient; and
b) determining the likelihood that the patient will develop symptoms of CIPN based on the assessment of step a).

24. The method of claim 23, further comprising determining electrochemical skin conductance with a system comprising an anode and a cathode, placed on different regions of the patient, and an adjustable DC source, which is controlled in order to feed the anode with a DC current.

25. The method of claim 24, wherein step a) comprises:
applying DC voltage pulses of varying voltage values to the anode for given durations allowing a stabilization of electrochemical phenomena in the patient in a vicinity of the anodes;
collecting data representative of current between the anode and the cathode, and of potentials of the anode and the cathode, for different DC voltages; and
from the data, computing data representative of the electrochemical skin conductance of the patient.

26. A method of prognosing CIPN in a cancer patient treated by chemotherapy, the method comprising:
a) measuring electrochemical skin conductance of the patient at a first time point;
b) measuring the electrochemical skin conductance of the patient at a second time point;
c) comparing the measurement of step a) and the measurement of step b); and
d) prognosing CIPN in the patient based on the comparison of step c).

27. The method of claim 26, further comprising determining the electrochemical skin conductance with a system comprising an anode and a cathode, placed on different regions of the patient, and a controlling DC source, to feed the anode with a DC current.

28. The method of claim 27, wherein step a) comprises:
applying DC voltage pulses of varying voltage pulses to the anode for given durations allowing a stabilization of electrochemical phenomena in the patient in a vicinity of the anode;
collecting data representative of the current between the anode and the cathode, and of potentials of the anode and the cathode, for different DC voltages; and
from the data, computing data representative of the electrochemical skin conductance of the patient.

29. A method for adapting a chemotherapy treatment in a cancer patient, wherein the method comprises:
a) measuring electrochemical skin conductance of the patient;
b) determining a likelihood that the patient will develop symptoms of CIPN based on the assessment of step a); and
c) adapting the chemotherapy treatment of the patient based on the likelihood of step b).

30. The method of claim 29, wherein the electrochemical skin conductance is determined with a system comprising an anode and a cathode, intended to be placed on different regions of the patient, and an adjustable DC source, which is controlled in order to feed the anode with a DC current.

31. The method of claim 30, wherein step a) comprises:
applying DC voltage pulses of varying voltage values to the anode for given durations allowing a stabilization of an electrochemical phenomena in the patient;
collecting data representative of the current between the anode and the cathode, and potentials of the anode and the cathode, for different DC voltages; and
from the data, computing data representative of the electrochemical skin conductance of the patient.

32. The method of claim 31, wherein step b) comprises comparing the electrochemical skin conductance of the patient with a reference value.

33. The method of claim 32, wherein the reference value is the electrochemical skin conductance of a healthy individual.

34. The method of claim 32, wherein the reference value is the electrochemical skin conductance of the patient at a second time point.

35. The method of claim 29, wherein the adaptation of step c) comprises reducing or suppressing the chemotherapy if the patient is assessed as being likely to develop symptoms of CIPN.

36. The method of claim 29, wherein the adaptation of step c) comprises continuing or increasing the chemotherapy if the patient is assessed as not being likely to develop symptoms of CIPN.
